(19) United States
(12) Patent Application Publication
(10) Pub. No.: US 2019/0304581 A1
(43) Pub. Date: Oct. 3, 2019

(54) SYMPTOM AND TRIGGER DATA TRACKING SYSTEM

(71) Applicants: Douglas James Leppard, Orlando, FL (US); Benjamin David Leppard, Torrance, CA (US)

(72) Inventors: Douglas James Leppard, Orlando, FL (US); Benjamin David Leppard, Torrance, CA (US)

(21) Appl. No.: 16/373,758

(22) Filed: Apr. 3, 2019

Related U.S. Application Data
(60) Provisional application No. 62/652,285, filed on Apr. 3, 2018.

(51) Int. Cl. G16H 15/00; G16H 20/60; G16H 10/65

(52) U.S. Cl. CPC G16H 15/00; G16H 10/65; G16H 20/60

(57) ABSTRACT

This invention tracks and collects the data of the symptoms a person is experiencing as the symptoms happen, on a symptom logger, and are converted into a symptom score. The data is sent wirelessly to a tracker mobile app running on a user device when they are in range of each other. The tracker mobile app shows logged symptoms by date and time and tracks and collects the data of what may be triggering those symptoms (e.g., food, drink, medication, etc.). The tracker mobile app also keeps a daily journal of heath questions and sends reminders for medications to be taken. All the data is sent continually to the cloud. The data from the cloud is put into graphical reports for the medical professional and the patient. By correlating the data of symptoms and triggers, the medical professional can determine what is causing the patients symptoms and prescribe possible solutions.

### CROSS-REFERENCES TO RELATED APPLICATIONS

This application claims the benefit of U.S. Provisional Application Ser. No. 62/652,285 filed on Apr. 3, 2018 titled “SYMPTOM AND TRIGGER DATA TRACKING SYSTEM” which is incorporated herein by reference in its entirety for all that is taught and disclosed therein.

### BACKGROUND

When a patient comes into the doctor's office and the medical professional asks a series of questions, this usually does not pinpoint what the real issues are. The medical professional often must guess what is happening with the patient. This process usually leaves the medical professional frustrated in obtaining the best information in order to provide the best diagnosis or advice. The medical professional may recommend the current methods of using daily logs for symptoms and/or journals to track food intake, but typically these methods are not detailed enough and are often not in electronic form, making it difficult to track and correlate the information in order to pinpoint what the real issues are. The data captured is not available to be used to make reports that correlate symptoms with health issues. What is needed in the art is a way to track and collect the data of the symptoms a person is experiencing, such as pain, itching, stress, etc., and track and collect the data of what may be triggering those symptoms, such as food consumed, medications, environmental issues, etc., and correlate the data of symptoms and triggers to show what is causing those symptoms or what relieves those symptoms.

### SUMMARY

The Symptom and Trigger Data Tracking System tracks and collects the data of the symptoms a person is experiencing, and tracks and collects the data of what may possibly be triggering those symptoms. The data of symptoms and triggers is correlated to show what is causing those symptoms, or, what is relieving those symptoms. The symptoms are recorded as they happen and are converted into a symptom score which can be entered into a database for further processing.

The Symptom and Trigger Data Tracking System has three components: the Symptom Logger, the Tracker Mobile Application for iOS and Android (hereinafter Tracker Mobile App), and the data collection in the cloud that can take the data from the Symptom Logger and Tracker Mobile Application to create reports for the medical professional and the patient.

The detailed description below describes the Symptom Logger, the Tracker Mobile App, and the data collection in the cloud. The solution described below enables the patient to press one of five different input buttons on the Symptom Logger when symptoms are felt. Up to five different symptoms can be recorded, or, a single symptom using five levels of effect. One skilled in the art will recognize that fewer than five input buttons or more than five input buttons could be used, but five input buttons are shown in one embodiment. The data is synchronized to the Tracker Mobile App when the Symptom Logger is in range with the smartphone, tablet, or other suitable device (herein after referred to as the user's device) running the Tracker Mobile App. Utilizing the Tracker Mobile App, the patient can view symptoms, log new symptoms, log potential triggers, answer daily health questions, receive reminders to take medications, log medications taken, and other useful information.

### BRIEF DESCRIPTION OF THE SEVERAL VIEWS OF THE DRAWINGS

FIG. 1 shows an embodiment of the Symptom Logger.

FIG. 2 shows the bottom of a custom designed circuit board in an embodiment of the Symptom Logger.

FIG. 3 shows the top of a custom designed circuit board in an embodiment of the Symptom Logger.

FIG. 4 shows the flowchart of transferring data from the Symptom Logger to the Tracker Mobile App in an embodiment of the Symptom Logger.

FIG. 5 shows the Welcome screen in an embodiment of the Tracker Mobile App.

FIG. 6 shows the Set-up Sensive App with Symptom Logger Screen in an embodiment of the Tracker Mobile App.

FIG. 7 shows the Device Linked Screen in an embodiment of the Tracker Mobile App.

FIG. 8 shows the Register Screen in an embodiment of the Tracker Mobile App.

FIG. 9 shows the Register flowchart in an embodiment of the Tracker Mobile App.

FIG. 10 shows the Login Screen in an embodiment of the Tracker Mobile App.

FIG. 11 shows the Login flowchart in an embodiment of the Tracker Mobile App.

FIG. 12 shows the Symptoms Log Screen in an embodiment of the Tracker Mobile App.

FIG. 13 shows the Add Symptom Screen in an embodiment of the Tracker Mobile App.

FIG. 14 shows the Symptoms flowchart in an embodiment of the Tracker Mobile App.

FIG. 15 shows the Triggers Log Screen in an embodiment of the Tracker Mobile App.

FIG. 16 shows the Triggers Entry Screen in an embodiment of the Tracker Mobile App.

FIG. 17 shows the Add Trigger Screen in an embodiment of the Tracker Mobile App.

FIG. 18 shows the Triggers flowchart in an embodiment of the Tracker Mobile App.

FIG. 19 shows the Journal Screen in an embodiment of the Tracker Mobile App.

FIG. 20 shows the New Journal Entry Screen in an embodiment of the Tracker Mobile App.

FIG. 21 shows the Journal flowchart in an embodiment of the Tracker Mobile App.

FIG. 22 shows the Reminder Pop-up Screen in an embodiment of the Tracker Mobile App.

FIG. 23 shows the Reminder Pop-up flowchart in an embodiment of the Tracker Mobile App.

FIG. 24 shows the Reminders List Screen in an embodiment of the Tracker Mobile App.

FIG. 25 shows the Reminder Edit Screen in an embodiment of the Tracker Mobile App.

FIG. 26 shows the Reminders flowchart in an embodiment of the Tracker Mobile App.

FIG. 27 shows the Settings Screen in an embodiment of the Tracker Mobile App.

FIG. 28 shows the Settings flowchart in an embodiment of the Tracker Mobile App.

FIG. 29 shows the Menu Tab Icons at the bottom of the Tracker Mobile App in an embodiment of the Tracker Mobile App.

FIG. 30 shows the Menu System flowchart in an embodiment of the Tracker Mobile App.

FIG. 31 shows the Sync Data to Cloud flowchart in an embodiment of the Tracker Mobile App.

FIG. 32 shows an embodiment of the environment in which the symptom and trigger data tracking system operates.

FIG. 33 shows a sample report tracking pain level against foods consumed in an embodiment of the symptom and trigger data tracking system.

FIG. 34 shows a sample report tracking pain level against following a diet in an embodiment of the symptom and trigger data tracking system.

To assist in the understanding of the present disclosure the following list of components and associated numbering found in the drawings is provided herein:

Table of Components.

| Component | # |
|---|---|
| Symptom Logger | 2 |
| Top case | 4 |
| Flexible cover | 6 |
| Input buttons | 8 |
| Custom designed circuit board | 10 |
| Bottom case | 12 |
| Bottom side | 14 |
| Battery holder | 16 |
| Connector | 18 |
| Capacitor | 20 |
| Communications module | 22 |
| Top side | 24 |
| Buzzer | 26 |
| Set Up Device button | 28 |
| Pick Your Device | 30 |
| Skip Linking button | 32 |
| Start Using Sensive button | 34 |
| Email Input Box | 36 |
| Password Input Box | 38 |
| Group Number Input Box | 40 |
| Register button | 42 |
| Login button | 44 |
| +sign | 46 |
| Symptom | 48 |
| Symptom Date | 50 |
| Symptom Time | 52 |
| Symptom options | 54 |
| +sign | 56 |
| Trigger date entry box | 58 |
| Trigger time entry box | 60 |
| Trigger radio buttons | 62 |
| Add new trigger item | 64 |
| Trigger name entry box | 65 |
| Add trigger button | 66 |
| Back arrow add trigger | 67 |
| +sign | 68 |
| Journal date entry box | 70 |
| Answer entry boxes | 72 |
| Medication | 73 |
| Taken button | 74 |
| Wait 15 minutes button | 76 |
| Ignore button | 78 |
| Medication | 79 |
| +sign | 80 |
| Reminder item | 82 |
| Reminder time | 84 |
| Back arrow reminder edit | 85 |
| Sync | 86 |
| Logout | 88 |
| Data synchronization | 90 |
| Send Reports | 92 |
| Application version | 94 |
| Battery level | 96 |
| Software version | 98 |
| Hardware version | 100 |
| Symptoms icon | 102 |
| Triggers icon | 104 |
| Journal icon | 106 |
| Reminders icon | 108 |
| Settings icon | 110 |
| User | 112 |
| User's device | 114 |
| Wireless connection | 116 |
| Wireless connection | 118 |
| Cloud | 120 |
| Communications link | 122 |
| Medical professionals | 124 |
| Pain level line | 126 |
| Pain values | 127 |
| Food list | 128 |
| Dots | 130 |
| Grid | 132 |
| Days | 134 |
| Pain level line | 136 |
| Pain values | 137 |
| Diet line | 138 |
| Days | 140 |
| Circles | 142 |
| Circle | 144 |
| Circles | 146 |

### DETAILED DESCRIPTION

Referring now to the Figures, like reference numerals and names refer to structurally and/or functionally similar elements thereof, and if objects depicted in the figures that are covered by another object, as well as the tag line for the element number thereto, may be shown in dashed lines.

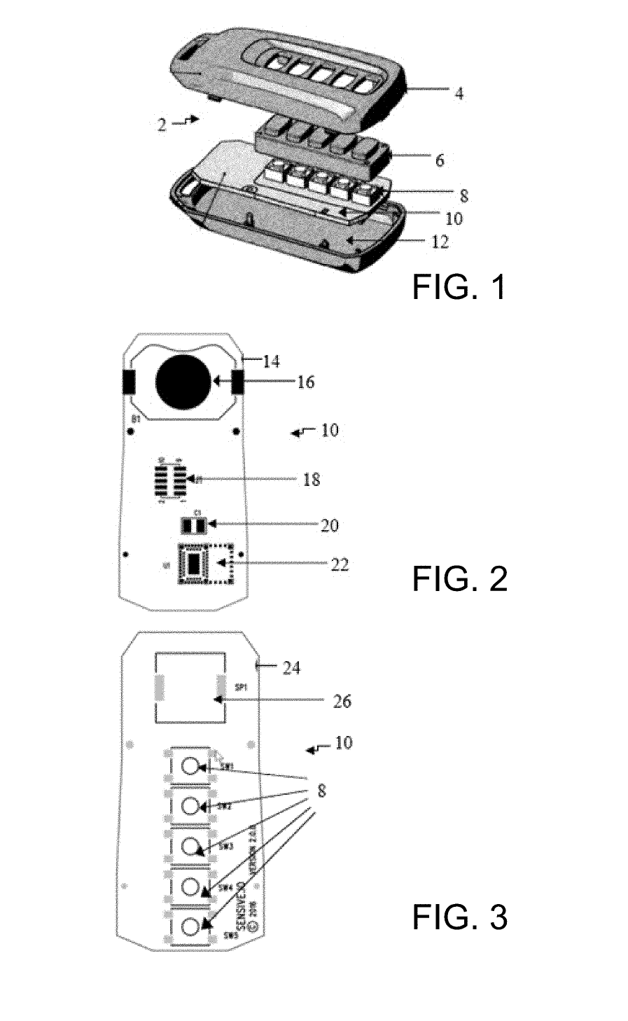

FIG. 1 shows an embodiment of the Symptom Logger. Referring now to FIG. 1, the Symptom Logger 2 in one embodiment is a Bluetooth® Key-FOB that has a top case 4 and a bottom case 12. In one embodiment, top case 4 and Bottom case 12 are made of polycarbonate but other materials may also be used. A cavity formed by top case 4 and bottom case 12 contains five input buttons 8 that are overlaid by a flexible cover 6 made of rubber, but other materials may also be used. Input buttons 8 have a tactile feel and when pressed, they make an electrical connection with a custom designed circuit board 10. Custom designed circuit board 10 can store months of data from pushes on each of the Input buttons 8, and transmit the stored data to the Tracker Mobile App running in the user's device via Bluetooth® when the Symptom Logger 2 comes within range of the user's device.

FIG. 2 shows the bottom side of the custom designed circuit board in an embodiment of the Symptom Logger. Referring now to FIG. 2, the bottom side 14 of the custom designed circuit board 10 contains a battery holder 16 for a battery (not shown in FIG. 2) which in one embodiment, is a CR2032 battery. A connector 18 allows a cable to be connected to custom designed circuit board 10 in order to program the onboard processor. Also shown on bottom side 14 is capacitor 20 and a communications module 22, which in one embodiment is an ISP130301 (nREF51) Bluetooth® Module with an integrated antenna for communicating with the user's device running the Tracker Mobile App.

FIG. 3 shows the top side of the custom designed circuit board in an embodiment of the Symptom Logger. Referring now to FIG. 3., top side 24 of the custom designed circuit board 10 contains a buzzer 26 for outputting sound, and the five input buttons 8. Each button press is stored as a data input in the Symptom Logger 2.

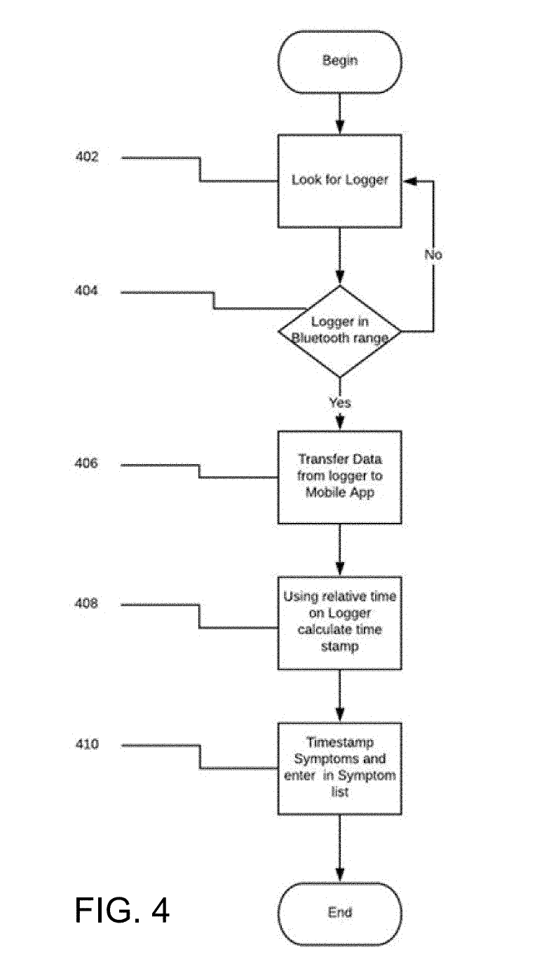

FIG. 4 shows the flowchart of transferring data from the Symptom Logger to the Tracker Mobile App in an embodiment of the Symptom Logger. Referring now to FIG. 4, in block 402 the communications module 22 within Symptom Logger 2 continually searches for the user's device that the Symptom Logger 2 is linked. In decision block 404, if the Symptom Logger 2 and the Tracker Mobile App running in the user's device are not in range of each other, control returns to block 402. If in decision block 404 the Symptom Logger 2 and the Tracker Mobile App running in the user's device are in range of each other, then in block 406 the data from the user input that has been stored in the Symptom Logger 2 is sent via Bluetooth® to the Tracker Mobile App.

In block 408 the Tracker Mobile App running in the user's device takes the data received from the Symptom Logger 2 and timestamps the data by looking at the relative time derived from the Symptom Logger 2. The Symptom Logger 2 cannot hold the real time of day, but it keeps its own internal time which is relative to the real time of day in the Tracker Mobile App running in the user's device. When the Tracker Mobile App syncs with the Symptom Logger 2, the Tracker Mobile App calculates the relative time to the Symptom Logger 2. From that the Tracker Mobile App can calculate a day and time (timestamp) for each recorded input button push, which could be either one of several symptoms, or, a level of effect of a symptom. In block 410 each timestamped symptom data is stored in the Tracker Mobile App and entered into a Symptom List. The flowchart shown in FIG. 4 repeats itself each time the Symptom Logger 2 and the Tracker Mobile App running in the user's device are in range of each other.

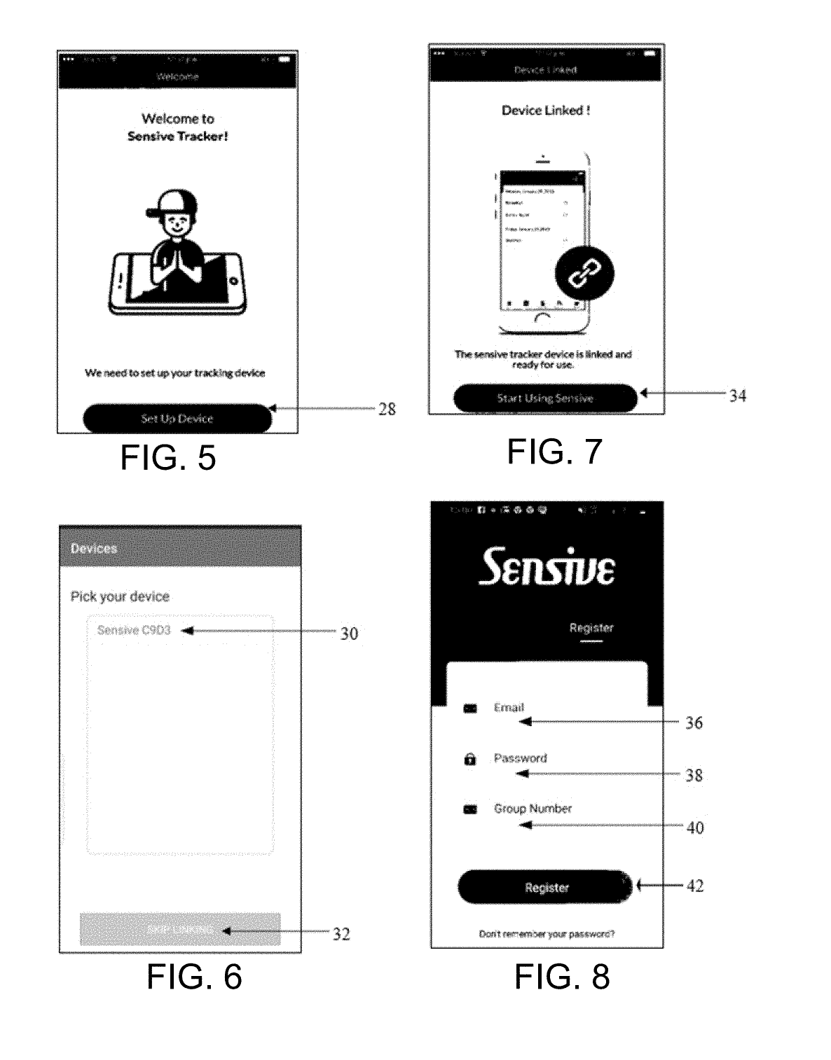

FIG. 5 shows the Welcome Screen in an embodiment of the Tracker Mobile App. Referring now to FIG. 5, at the bottom of the Welcome Screen is the Set-Up Device button 28 the user selects in order to set up the Tracker Mobile App with the Symptom Logger 2 that it is to be linked.

FIG. 6 shows the Set-up Sensive App with Symptom Logger Screen in an embodiment of the Tracker Mobile App. Referring now to FIG. 6, the user selects from a list of Symptom Loggers 2 that are within Bluetooth® range by selecting Pick Your Device 30 shown on the screen. If more than one family member is using a Symptom Logger 2 both devices may be detected and each user must select the one that they are to be linked. The user can select Skip Linking button 32 to if the user's device has already been linked to the Symptom Logger 2.

FIG. 7 shows the Device Linked Screen in an embodiment of the Tracker Mobile App. Referring now to FIG. 7, when the user selects the Pick Your Device 30 shown if FIG. 6, the screen shown in FIG. 7 indicates that the Symptom Logger 2 selected is successfully linked to the Tracker Mobile App running in the user's device. The user selects Start Using Sensive button 34 that brings up the Register Screen shown in FIG. 8.

FIG. 8 shows the Register Screen in an embodiment of the Tracker Mobile App. Referring now to FIG. 8, the user enters their email address in Email Input Box 36, enters a password in Password Input Box 38, and enters a group number in Group Number Input Box 40. The group number is given to the user by the medical professional who will be monitoring the user. The group number determines what symptoms, or what level of effect of a system, will be monitored, and are tied to the five Input buttons 8 (see FIGS. 12-14 below). A group is a concept that allows the Symptom Logger 2 and the Tracker Mobile App to be configured according to the medical provider's needs. Each group changes the mobile the following ways:

(1) The symptom list is changed so different symptoms can be tracked.

(2) The trigger list is changed per that group. The trigger list is preloaded by the needs of that group. Each user (patient) can add their own items on the trigger list.

(3) The Journal questions are set per that group. Each group can ask any number of questions and can have open ended questions and/or multiple-choice questions.

Each group has a group owner which in most cases will be the medical provider. A medical provider can own multiple groups for various types of groups. The group owner can order reports of its members.

Referring again to FIG. 8, the user selects the Register button 42 after entering their email, password, and group number to complete the Registration process.

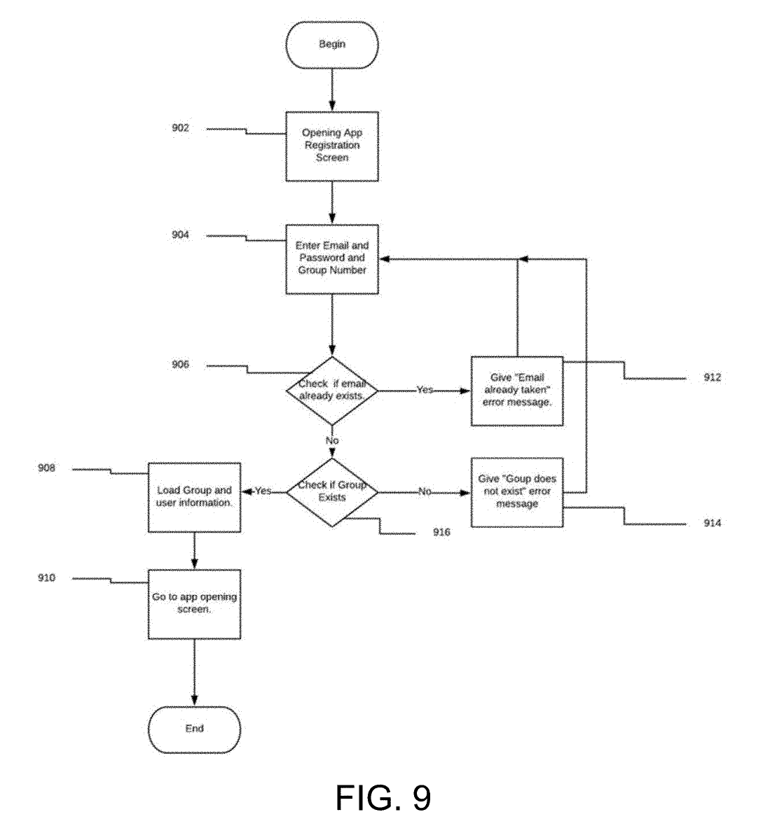

FIG. 9 shows the Register flowchart in an embodiment of the Tracker Mobile App. Referring now to FIG. 9, in block 902 the Register Screen opens to prepare for the new user. In block 904 the user enters their email, their chosen password, and group number. In decision block 906 the email entered is checked to see if that email already exists on the system stored in the cloud. Cloud services maybe provided by Amazon Web Services or Google Cloud or similar cloud services. If the email entered is already in use, then in block 912 an error message is displayed on the input screen and control returns to block 904 where the user can enter in a different email address. If decision block 906 determines that the email is available, then decision block 916 determines if the group number entered by the user is valid. If not, then in block 914 an error message is displayed on the input screen and control returns to block 904 where the user can enter a valid group number. If decision block 916 determines that the group number entered by the user is valid, then in block 908 the group information and user information is stored into the Tracker Mobile App. In block 910 the Tracker Mobile App opening screen is displayed and the Registration ends.

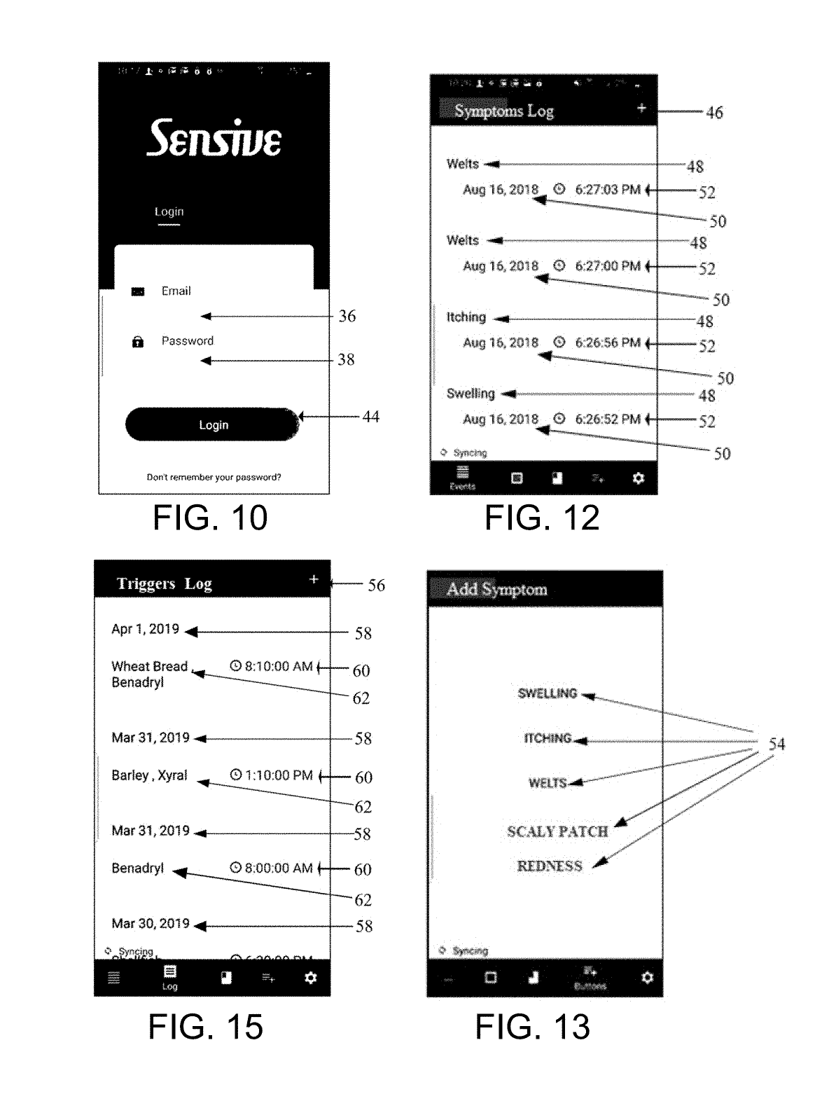

FIG. 10 shows the Login Screen in an embodiment of the Tracker Mobile App. Referring now to FIG. 10, if the user is registered, then the Login Screen is displayed and the user enters their email into Email Input Box 36 and their password into Password Input Box 38. The user then selects the Login button 44 which takes the user to the Symptoms Log Screen shown in FIG. 12.

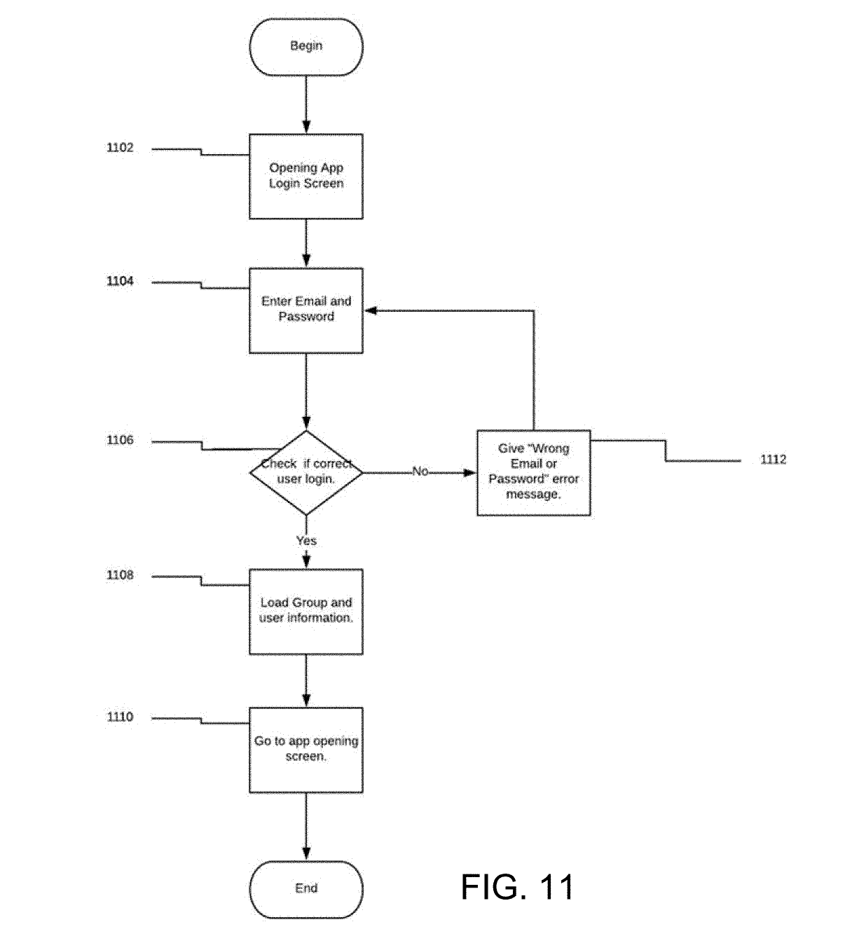

FIG. 11 shows the Login flowchart in an embodiment of the Tracker Mobile App. Referring now to FIG. 11, in block 1102 the opening Login Screen is displayed to allow the user to enter their login information. In block 1104 the user enters their email and password. Decision block 1106 checks the user entered information against the database of user information to determine if the entered information is correct. If the entered user email and/or password does not agree with database, then in block 1112 an error message is given to the user that a bad email and/or bad password has been entered, and control returns to block 1104 where the user can try again to enter in correct information. If decision block 1106 determines that the entered user email and password agree with the information stored in the database is correct, then in block 1108 the user group and user information is loaded and in block 1110 the opening screen is displayed and the Login process ends.

FIG. 12 shows the Symptoms Log Screen in an embodiment of the Tracker Mobile App. Referring now to FIG. 12, the Symptoms Log Screen shows the symptoms 48 that the user has logged. In this example, the symptoms 48 that have been logged include welts, itching, and swelling. For each logged symptom 48, there is an associated Symptom Date 50 and a Symptom Time 52. The symptoms log is the display of the data collected from the Symptom Logger 2. If the user wants to add a new symptom that is available in the group, the user selects+sign 46 which displays the Add Symptom Screen shown in FIG. 13.

FIG. 13 shows the Add Symptom Screen in an embodiment of the Tracker Mobile App. Referring now to FIG. 13, the symptom options 54 correspond to the 1-5 input buttons 8 on the Symptom Logger 2. As shown in FIG. 12, three symptoms 48 have been logged: swelling tied to button 1, itching tied to button 2, and welts tied to button 3. FIG. 13 shows that two other symptoms, scaly patch tied to button 4, and redness tied to button 5, are also members of the group that can be added by the user. To add scaly patches as a symptom to be monitored, the user selects symptom option 54 for scaly patch and the Symptoms Log Screen of FIG. 12 is displayed. Now, when input button 4 on Symptom Logger 2 is pressed, scaly patch symptom 48 will be displayed along with a Symptom Date 50 and a Symptom Time 52.

The symptom and trigger data tracking system can be used to help treat many different conditions. For example, 90% of people with Lupus have joint pain. Joint pain is a symptom that can be tracked with Symptom Logger 2. Possible triggers are food eaten which may express itself in joint pain. The inventor had his Lupus cured by utilizing Symptom Logger 2 to track his symptoms and triggers and the analysis of the reports that correlated the symptoms with the triggers.

In another example, most hives patients have the symptoms of itching, welts, swelling and blanching. The most common foods that can trigger hives include milk, eggs, wheat, soy, fish, shellfish, peanuts, and tree nuts. Other triggers include stress, heat, and animals such as dogs and cats. Correlating the symptoms with the possible triggers can determine for each individual which triggers are dominant with them. One hive sufferer may have completely different triggers from another hive sufferer. Linking the specific symptom with the specific trigger(s) can lead to cessation of the condition.

Headache or migraines are a symptom that can occur along with nausea, vomiting, and extreme sensitivity to light and sound. Headache or migraines can be triggered by hormonal changes, aged cheeses, salty foods, processed foods, food additives, alcohol—especially wine, highly caffeinated beverages, stress, missing sleep, change of weather, and more. Correlating the symptoms with the possible triggers can help an individual learn to avoid those triggers which are most problematic to them. Many other diseases or conditions may also be tracked and evaluated, such as arthritis, respiratory ailments, gastrointestinal conditions, etc.

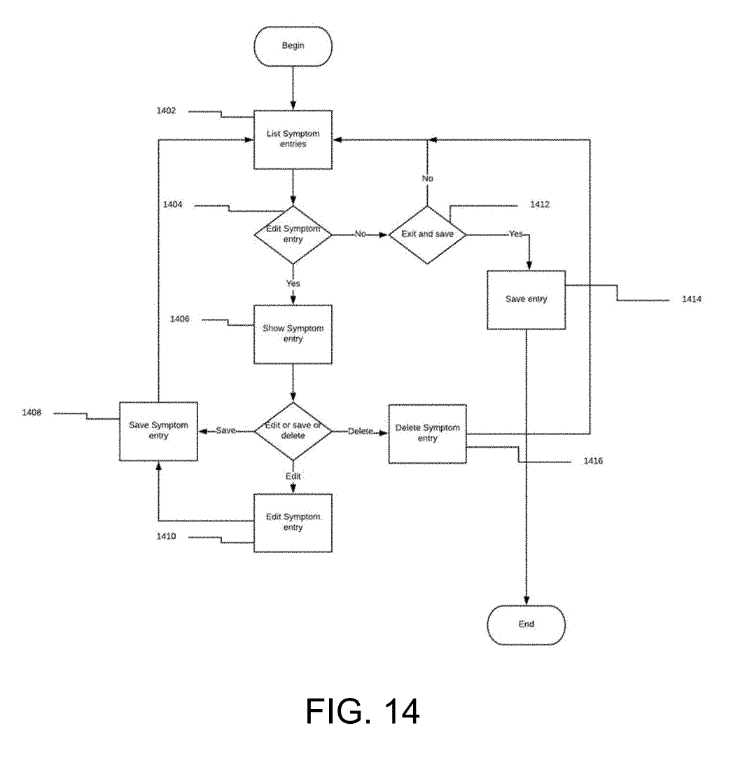

FIG. 14 shows the Symptoms flowchart in an embodiment of the Tracker Mobile App. Referring now to FIG. 14, block 1402 displays a list of symptom log entries that have been entered by the user utilizing the Symptom Logger 2 up to this point in time. Decision block 1404 determines if the user wants to add a new symptom to begin tracking. If no, then the symptoms process end. If yes, in block 1406 the list of five symptoms are displayed. Decision block 1408 determines if the user selects a symptom to begin monitoring. If no symptom is selected, control returns to block 1402. If the user has selected a symptom to begin monitoring, then in block 1410 the symptom selected is added to the symptom log along with a timestamp, and control returns to block 1402 which displays the list of symptom log entries.

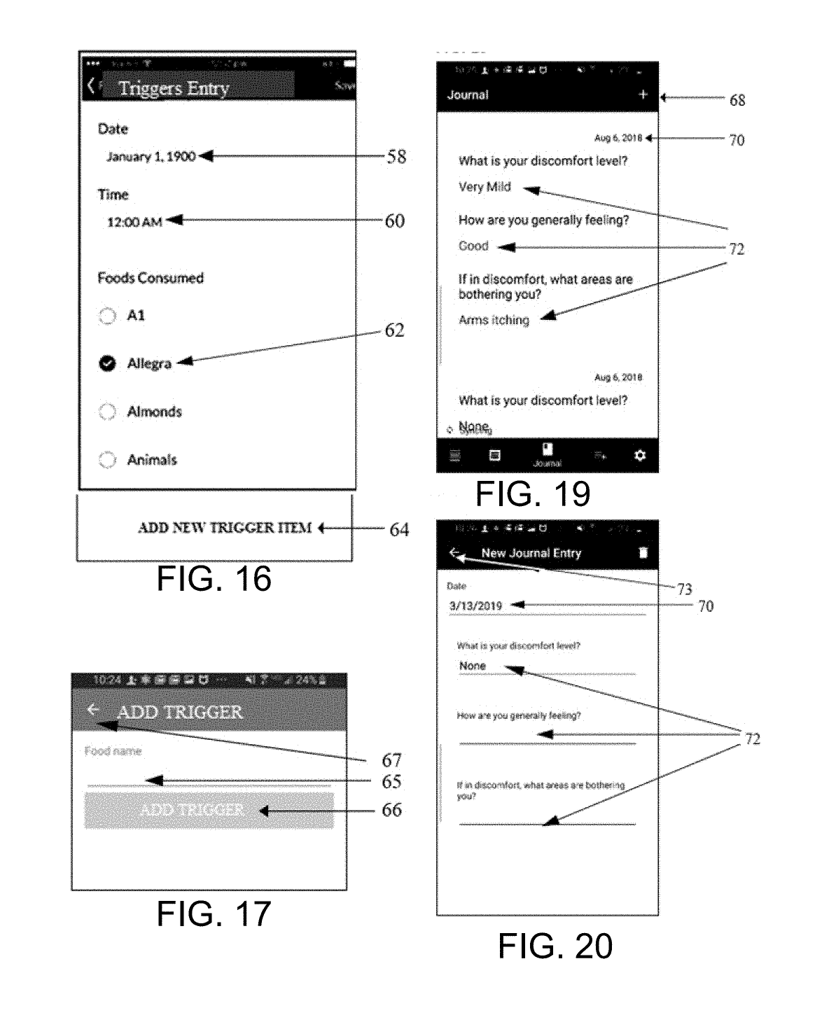

FIG. 15 shows the Triggers Log Screen in an embodiment of the Tracker Mobile App. Referring now to FIG. 15, displayed are the user data of potential triggers the user logged in the Triggers Entry Screen shown in FIG. 16. Each symptom contains a trigger date 58, a trigger time 60, and the particular trigger 62 selected from a potential list of triggers. A medical professional establishes a beginning list of triggers that the medical professional deems to be the most relevant. The user can add a trigger entry by selecting the +sign 56 which displays the Triggers Entry Screen shown in FIG. 16.

FIG. 16 shows the Triggers Entry Screen in an embodiment of the Tracker Mobile App. Referring now to FIG. 16, the user can add a trigger date in a trigger date entry box 58, a trigger time in a trigger time entry box 60, and a trigger from the list of triggers by selecting a trigger radio button 62 in front of the trigger name. The Tracker Mobile App automatically shows the current date and time, but these can be changed if the user is referring to an event in the past. For example, if a person had lunch and left their Symptom Logger 2 in their office, they could enter the food they ate upon returning to their office, and the user would change the time to when they actually ate. To add a new trigger item to the list of triggers, the user selects add new trigger item 64 which displays the Add Trigger Screen shown in FIG. 17.

FIG. 17 shows the Add Trigger Screen in an embodiment of the Tracker Mobile App. Referring now to FIG. 17, the user enters the new trigger name in trigger name entry box 65 and selects add trigger button 66 which will automatically add the new entry to the list of triggers shown in FIG. 16. To save the record, the user selects the back arrow add trigger 67, and the Triggers Log Screen shown in FIG. 15 is displayed.

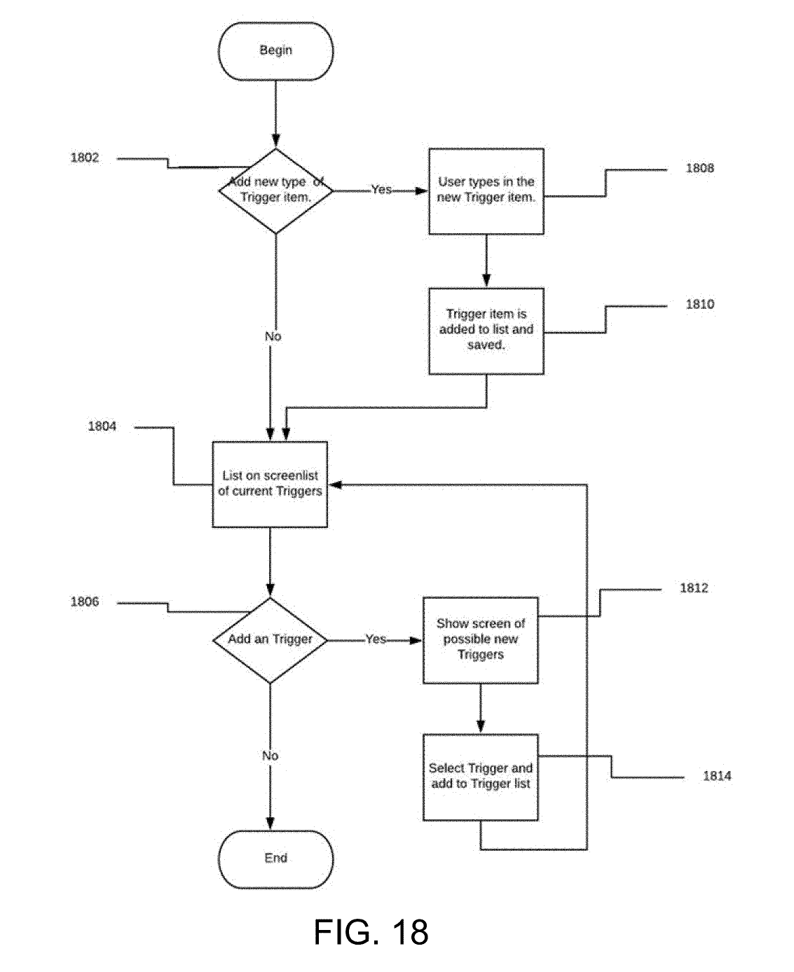

FIG. 18 shows the Triggers flowchart in an embodiment of the Tracker Mobile App. Referring now to FIG. 18, block 1802 displays the current list of triggers. Decision block 1804 determines if the user has selected to add a new type of trigger item to the current list of triggers. If yes, then in block 1808 the user types in the new trigger item. In block 1810 the new trigger item is added to the list of triggers and is saved. Control then returns to block 1802 which displays the current list triggers. If decision block 1804 determines that the user has not selected to add a new type of trigger item to the current list of triggers, then decision block 1806 determines if the user has opted to add an existing trigger from the list of triggers being monitored. If no, the triggers process ends. If yes, then block 1812 displays the preexisting list of possible new triggers. In block 1814 the user chooses a new trigger from the list of triggers to be added to the monitored triggers. Control then returns to block 1802 which displays the current list triggers being monitored.

FIG. 19 shows the Journal Screen in an embodiment of the Tracker Mobile App. Referring now to FIG. 19, the Journal date 70 of the entry is displayed along with answers to journal questions 72. To add a new journal entry the user selects+sign 68 and the New Journal Entry Screen is displayed as shown in FIG. 20.

FIG. 20 shows the New Journal Entry Screen in an embodiment of the Tracker Mobile App. Referring now to FIG. 20, the current date is automatically populated in journal date entry box 70, but can be changed by the user by typing in a new date in journal date entry box 70. The user answers the journal questions by typing in the answers in answer entry boxes 72. To save the journal entry, the user selects the back arrow new journal entry 73, and the Journal Screen shown in FIG. 19 is displayed. In one embodiment, the user can only enter one journal entry per day.

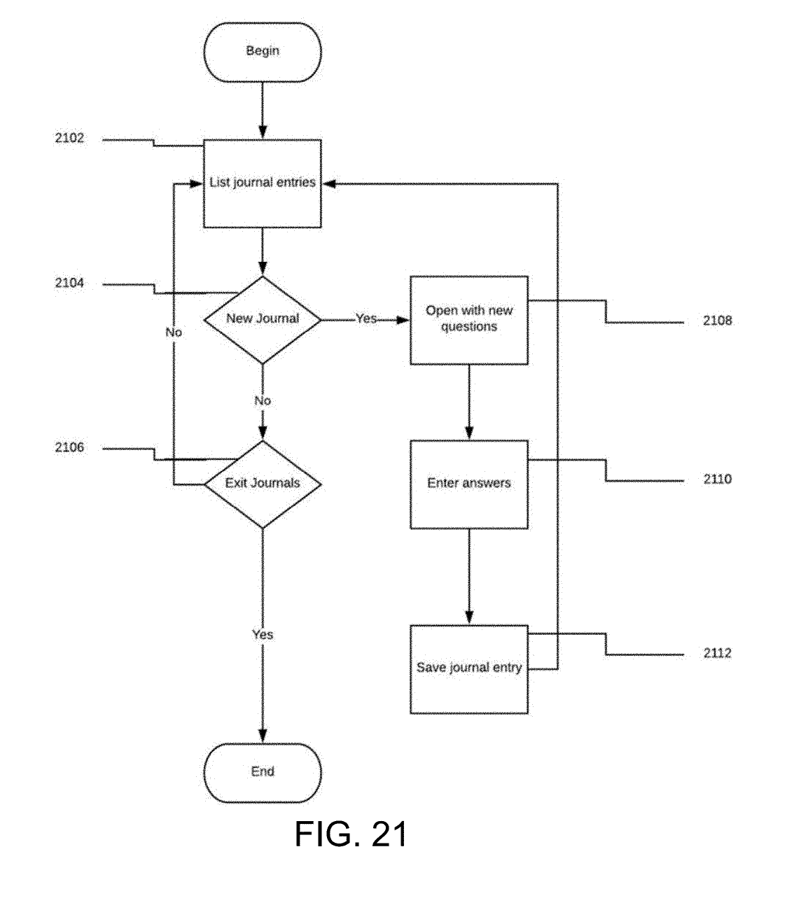

FIG. 21 shows the Journal flowchart in an embodiment of the Tracker Mobile App. Referring now to FIG. 21, block 2102 displays the list of journal entries made to date. Decision block 2104 determines if the user has selected to make a new journal entry. If no, then decision block 2106 determines if the user has selected to exit making a journal entry. If yes, the Journal process ends. If no, control returns to block 2102.

If decision block 2104 determines that the user has selected to make a new journal entry, then in block 2108 a screen is displayed to allow the user to answer journal questions. In block 2110 the user enters the answers to the journal questions displayed. In block 2112 the journal entries entered by the user are saved and control is returned to block 2102 where the new journal entry is displayed on the Journal Screen.

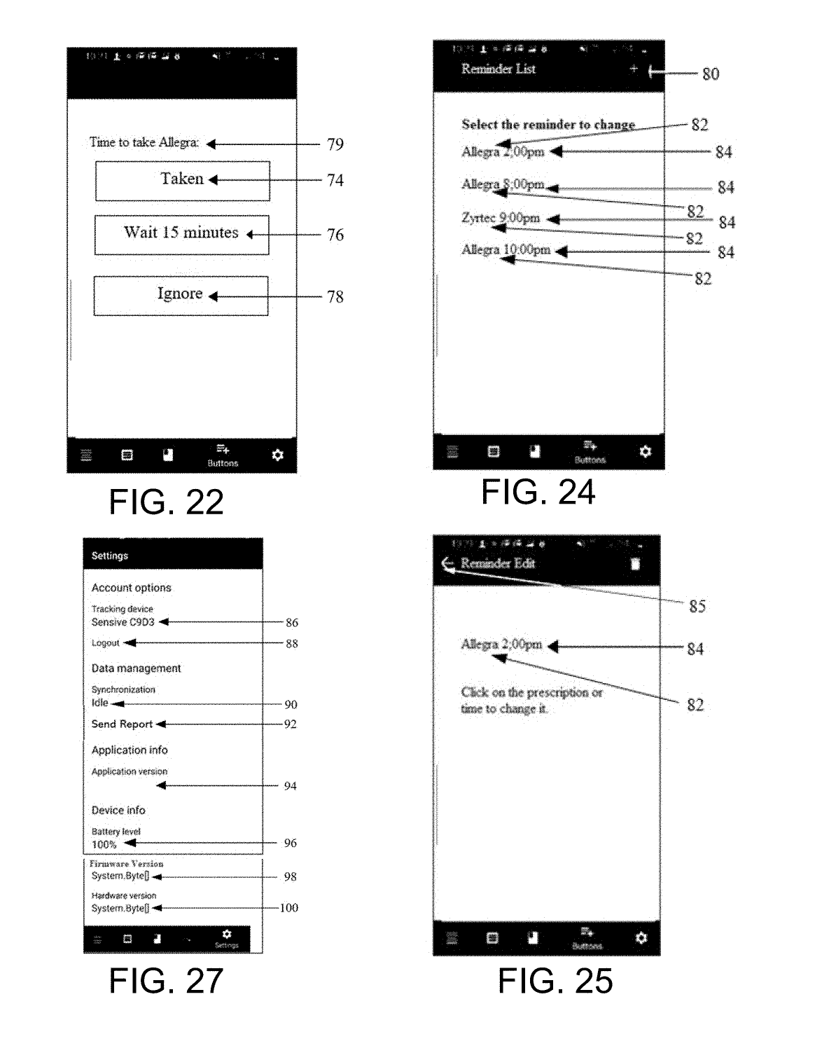

FIG. 22 shows the Reminder Pop-up Screen in an embodiment of the Tracker Mobile App. Referring now to FIG. 22, the Reminder Pop-up Screen displays on the user's device according to a predetermined schedule. The Reminder shows what medication 79 (a prescription or an over-the-counter medication) the user is supposed to take at the scheduled time. If the user takes the medication, the user selects the taken button 74 and an entry is added to the log. The user can select the wait 15 minutes button 76 and the user will be reminded again in 15 minutes to take the medication. The user can also select the ignore button 78 and not take the medication.

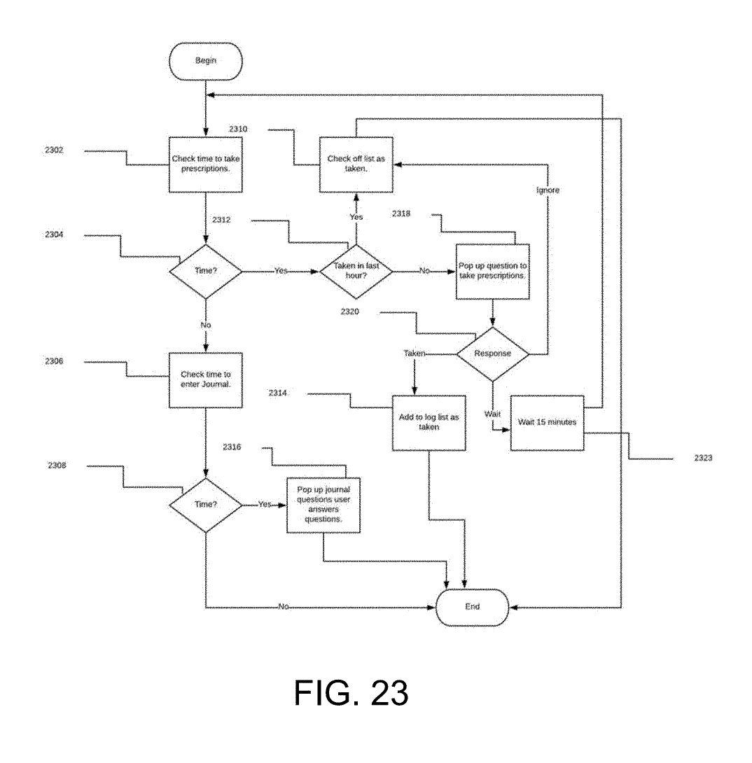

FIG. 23 shows the Reminder Pop-up flowchart in an embodiment of the Tracker Mobile App. Referring now to FIG. 23, in block 2302 the database checks to see if it is time to take a medication. If decision block 2304 determines that it is not time for the user to take a medication, block 2306 checks the database to see if it is time to do a journal entry. If no, the Reminder Pop-up process ends. If yes, in block 2316 the journal questions are displayed and the user enters in the responses to the journal questions and the Reminder Pop-up process ends.

If decision block 2304 determines that it is time for the user to take a medication, decision block 2312 checks to see if the medication has been taken by the user within the last hour ahead of the scheduled time. If yes, in block 2310 the medication taken is checked off the list and shown as being taken and the Reminder Pop-up process ends.

If decision block 2312 determines that the medication has not been taken by the user within the last hour, block 2318 displays the pop-up reminder to take the medication. Decision block 2320 determines if the user has selected one of three possible responses: taken the medication; wait 15 minutes to take the medication; or ignore taking the medication. If decision block 2320 determines the user has taken the medication, then block 2314 adds to the log list that the medication has been taken and the Reminder Pop-up process ends. If decision block 2320 determines the user has selected to wait 15 minutes, then in block 2323 the Reminder Pop-up process suspends for 15 minutes and control returns to block 2302. If decision block 2320 determines the user has chosen to ignore taking the medication, then in block 2310 the medication is checked off the list and shown as not being taken and the Reminder Pop-up process ends.

FIG. 24 shows the Reminder List Screen in an embodiment of the Tracker Mobile App. Referring now to FIG. 24, a list of reminder items 82 are displayed along with a reminder time 84 for each reminder item 82. The user can edit a reminder item 82 by selecting the +sign 80 that displays a Reminder Edit Screen as shown in FIG. 25.

FIG. 25 shows the Reminder Edit Screen in an embodiment of the Tracker Mobile App. Referring now to FIG. 25, the user can edit the reminder item 82 and/or the reminder time 84 by clicking on the reminder item 82 and/or the reminder time 84 and typing in the new values. To save the edited reminder item 82 and/or the reminder time 84 the user selects back arrow reminder edit 85 that causes the Reminder List Screen shown in FIG. 24 to be displayed.

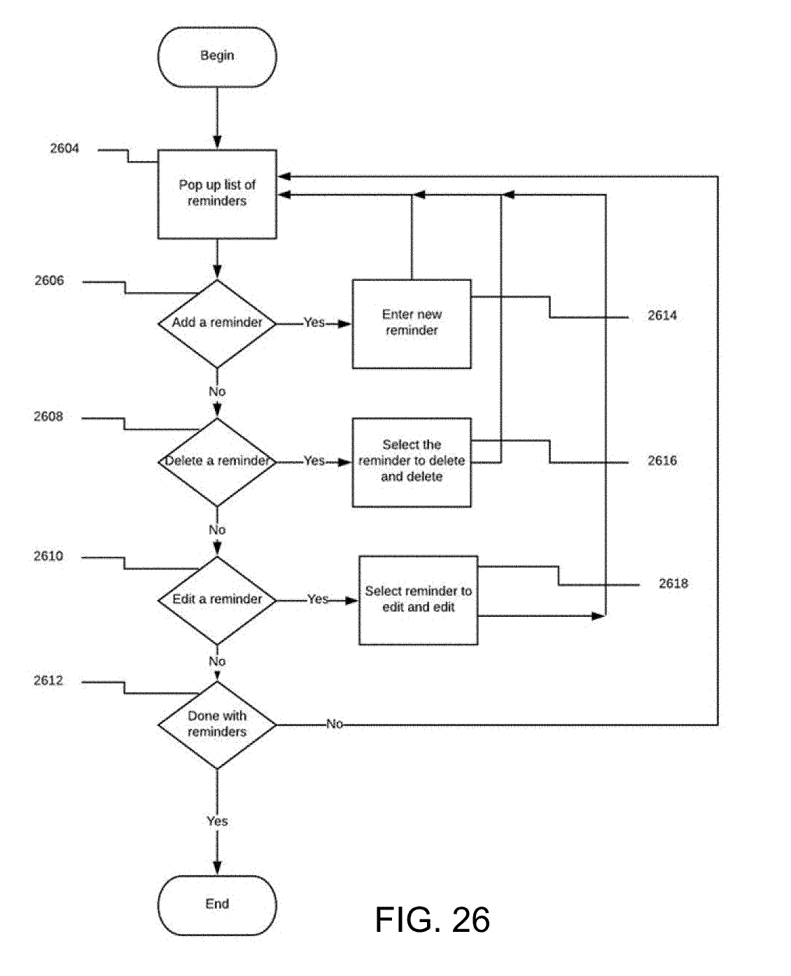

Screens similar to those shown in FIGS. 24 and 25 allow for a user to add new reminder items and to delete existing reminder items, as explained in FIG. 26 below.

FIG. 26 shows the Reminder Edit flowchart in an embodiment of the Tracker Mobile App. Referring now to FIG. 26, block 2604 displays the list of reminders. Decision block 2606 determines if the user has selected a reminder item to add. If yes, then in block 2614, the user enters the new reminder item information (medication and time to take the medication) and control returns to block 2604.

If decision block 2606 determines that the user has not selected a reminder item to add, then decision block 2608 determines if the user has selected to delete a reminder item. If yes, then in block 2616 the user selects the reminder item to delete and it is deleted and control returns to block 2604.

If decision block 2608 determines that the user has not selected a reminder item to delete, then decision block 2610 determines if the user has selected to edit a reminder item. If yes, then in block 2618 the user edits the reminder item selected and it is saved and control returns to block 2604.

If decision block 2610 determines that the user has not selected a reminder item to edit, then decision block 2612 determines if the user is done adding, deleting, or editing. If no, control returns to block 2604. If yes, the Reminder Edit process ends.

FIG. 27 shows the Settings Screen in an embodiment of the Tracker Mobile App. Referring now to FIG. 27, account options for the Symptom Logger 2 are displayed. Sync 86 displays the ID number of the Symptom Logger 2 that is unique to each Symptom Logger 2 and that has been synchronized with the Tracker Mobile App running in the user's device. Data synchronization 90 shows the current status of the synchronization process, which can either be idle or synchronizing. Battery level 96 displays the current battery level for the Symptom Logger 2. Application version 94 displays the application version of the Tracker Mobile App running in the user's device. It also includes Send Reports 92 which allows the user to select a report to send and who to send it to, and Logout 88 which allows the user to logout of the Tracker Mobile App. Also displayed is Application version 98 that displays the version number of the firmware (program) that runs on the Symptom Logger 2. Hardware version 100 displays the current hardware version of the Symptom Logger 2.

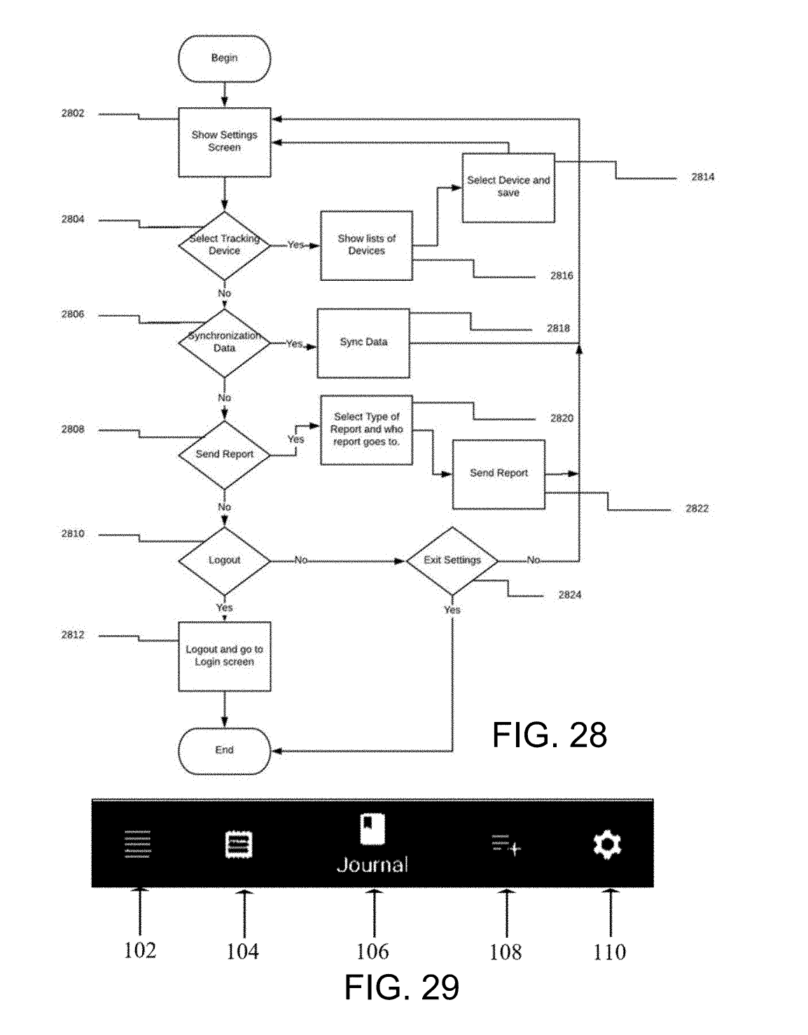

FIG. 28 shows the Settings flowchart in an embodiment of the Tracker Mobile App. Referring now to FIG. 28, block 2802 displays the settings screen and options to change those settings. Decision block 2804 determines if the user has clicked on select tracking device to choose the Symptom Logger 2 to be synchronized with the Tracker Mobile App. If yes, block 2816 displays the list of devices available to be synced with the Tracker Mobile App. In block 2814 the user selects the device to sync and the selection is saved. Control then returns to block 2802.

If decision block 2804 determines that the user has not clicked on select tracking device, then decision block 2806 determines if the user has selected to synchronize data to settings. If yes, block 2818 synchronizes the data in the Tracker Mobile App to the cloud. This is done automatically but this option allows the user to do it manually. Control then returns to block 2802.

If decision block 2806 determines that the user has not selected to synchronize data to settings, decision block 2808 determines if the user has selected to send a report. If yes, in block 2820 the user selects the type of report to send and who the report is to be sent. Block 2822 sends the report via email. Control then returns to block 2802.

If decision block 2808 determines that the user has not selected to send a report, decision block 2810 determines if the user has selected to logout of the Tracker Mobile App. If no, decision block 2824 determines if the user selects to exit the settings screen. If no, control then returns to block 2802. If yes, the Settings process ends.

If decision block 2810 determines that the user has selected to logout of the Tracker Mobile App, in block 2812 logout is completed and the login screen is displayed, thus ending the Settings process.

FIG. 29 shows the Menu Tab Icons at the bottom of the Tracker Mobile App in an embodiment of the Tracker Mobile App. Referring now to FIG. 29, the icons displayed help the user to know the area they are in and to navigate to other menu items. The Menu Tab Icons are symptoms icon 102, triggers icon 104, journal icon 106, reminders icon 108, and settings icon 110.

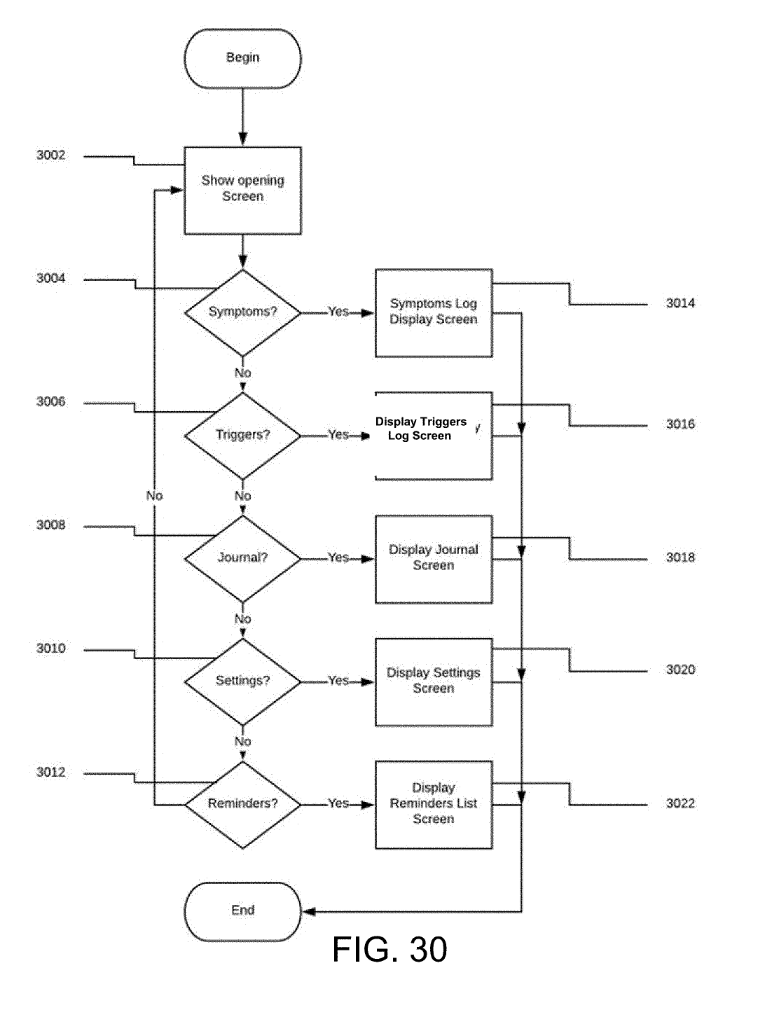

FIG. 30 shows the Menu System flowchart in an embodiment of the Tracker Mobile App. Referring now to FIG. 30, in block 3002 the opening screen for the Tracker Mobile App is displayed. Decision block 3004 determines if the user has selected the symptoms icon 102. If yes, block 3014 displays the Symptoms Log Screen (see FIG. 12) and the Menu System process ends. If no, control passes to decision block 3006.

Decision block 3006 determines if the user has selected the triggers icon 104. If yes, block 3016 displays the Triggers Log Screen (see FIG. 15) and the Menu System process ends. If no, control passes to decision block 3008.

Decision block 3008 determines if the user has selected the journal icon 106. If yes, block 3018 displays the Journal Screen (see FIG. 19) and the Menu System process ends. If no, control passes to decision block 3010.

Decision block 3010 determines if the user has selected the settings icon 110. If yes, block 3020 displays the Settings Screen (see FIG. 27) and the Menu System process ends. If no, control passes to decision block 3012.

Decision block 3012 determines if the user has selected the reminders icon 108. If yes, block 3014 displays the Reminders List Screen (see FIG. 24) and the Menu System process ends. If no, control returns to block 3002.

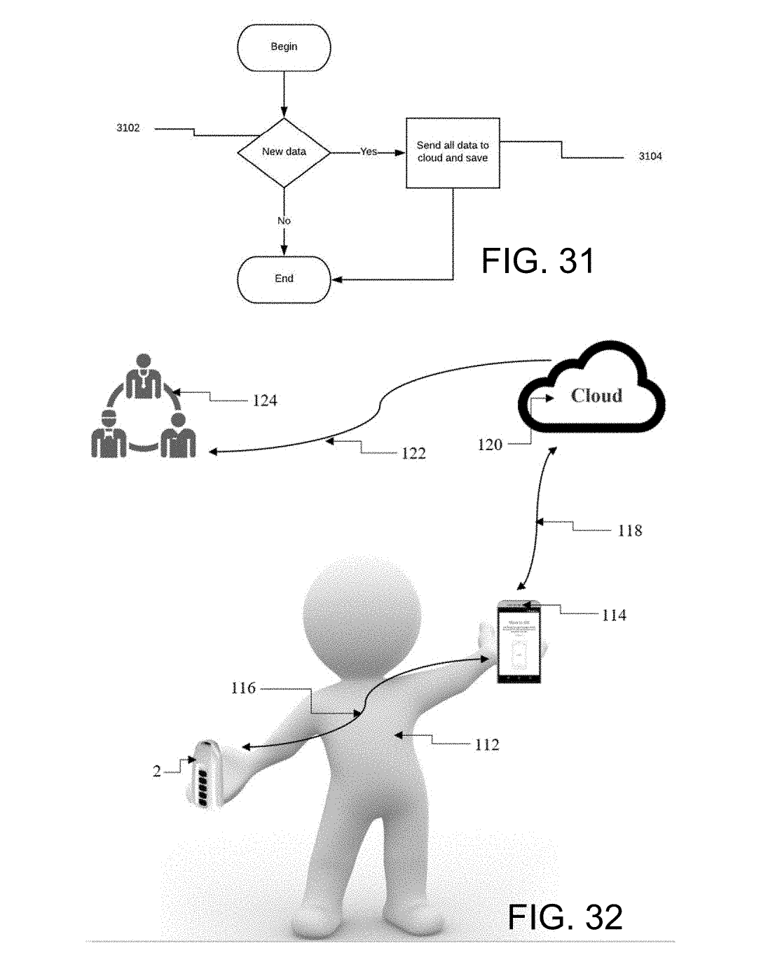

FIG. 31 shows the Sync Data to Cloud flowchart in an embodiment of the Tracker Mobile App. Referring now to FIG. 31, decision block 3102 determines if there is new data to be synced to the cloud. If yes, block 3104 sends all the new data not synced to the cloud where it is also stored in the cloud, and the Sync Data to Cloud process ends. If decision block 3102 determines that there is no new data to sync, then the Sync Data to Cloud process ends.

FIG. 32 shows an embodiment of the environment in which the symptom and trigger data tracking system operates. Referring not to FIG. 32, user 112 carries Symptom Logger 2 with him/her as he/she goes about their daily routines. There may be hundreds of users 112 each utilizing their own Symptom Logger 2 in conjunction with their own user device 114. Only one user 112 is shown in FIG. 32 for simplicity of presentation.

User 112 presses input buttons 8, (1-5) as symptoms are felt. User 112 may not always have their user device 114 in proximity to Symptom Logger 2. Therefore, Symptom Logger 2 stores data input until such time as Symptom Logger 2 comes into proximity to user device 114, at which time the data stored in Symptom Logger 2 is transferred by communications module 22 via wireless connection 116 to user's device 114. The data stored in the user's device 114 is sent to the cloud 120 via communications link 118. Communications link 118 may be any suitable communications channel, such as cellular telephone transmission, the Internet, LAN's, etc. Whenever the Tracker Mobile App is running in the user's device 114, and the user's device 114 has communications link 118, the Tracker Mobile App transfers the data automatically to the cloud 120. This makes the data collected basically “live” at all times unless the Symptom Logger 2 is out of range or the user's device 114 does not have communications link 118. User's device 114 are almost always connected to communications link 118. A database maintained in the cloud 120 stores information for all users 112.

Medical professionals 124 receive the data stored in the cloud 120 via communications link 122. Medical professionals 124 monitor users 112 through the data received from the cloud 120. Medical professionals 124 can request reports that are generated in the cloud 120 and are delivered to the medical professionals 124 via email through communications link 122. User 112 can also request reports utilizing the Mobile Tracker App running in the user's device 114 via Communications link 118 to in the cloud 120 and the reports are delivered via email through Communications link 118 to User 112. In one embodiment, Medical professionals 124 can send messages to the user's device 114, and output to the User 112 through the Mobile Tracker App running in the user's device 114.

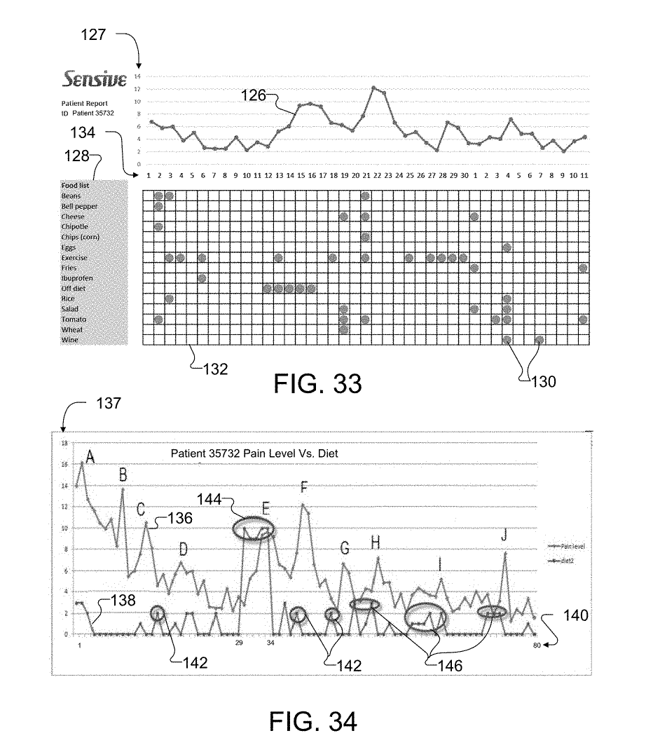

FIG. 33 shows a sample report tracking pain level against foods consumed in an embodiment of the symptom and trigger data tracking system. Referring now to FIG. 33, a user 112 suffering from Lupus used Symptom Logger 2 to track their pain level line 126 over a forty-one-day period against foods consumed as shown in food list 128. Each dot 130 in the grid 132 represents a food item from the trigger list for the Lupus group consumed on a particular day 134 as input by the user through the Mobile Tracker App running in the user's device 114. Pain level line 126 is the average pain level per hour for that day. The pain values 127 ranging from 0 to 14 are the total of buttons 1-5 pushed on Symptom Logger 2 and averaged out for the day per hour. Button 1 is worth 1 point, button 2 is worth 2 points, button 3 is worth 3 points, button 4 is worth 4 points, and button 5 is worth 5 points. The pain levels are 1=no pain, 2=low pain, 3=moderate pain, 4=heavy pain, and 5=severe pain. A lower averaged score indicates a lower pain level for the day. A score in the 2-3 range indicates that the user 112 is feeling pretty good. A score of 4 and above indicates that the user 112 is starting to feel bad.

As can be seen in FIG. 33, the days the user 112 was off diet on days 12-16, there is a corresponding jump in the pain level line 126. Also observed is the combination of foods consumed on day 21 also led to a spike in pain level line 126 that had an effect for several days afterward.

FIG. 34 shows a sample report tracking pain level against following a diet in an embodiment of the symptom and trigger data tracking system. Referring now to FIG. 34, a user 112 suffering from Lupus used Symptom Logger 2 to track their pain level line 136 over an eighty-day period against a diet, represented by diet line 138, prescribed by a medical professional 124. The pain values 137 ranging from 0 to 18 are the total of buttons 1-5 pushed on Symptom Logger 2 and averaged out for the day per hour as explained above in reference to FIG. 33. Diet line 138 represents how well user 112 held to the prescribed diet. Using the Mobile Tracker App running in the user's device 114, the user 112 would enter a score for keeping to the diet. A score of 0 represented that the user 112 kept to the prescribed diet perfectly. A score of 1 or 2 indicated that one or two items consumed were off diet for one meal, and those items were documented. A 9 or 10 score meant that the user 112 went off the diet totally for that day. User 112 purposefully went off the diet for a five-day test period from days 29-34. The report shows that when user 112 kept to the diet the pain level was reduced, and the times user 112 got off the diet the pain levels increased.

It can be observed from the report that it takes about 24-48 hours after eating something off-diet, which in this diet were identified as inflammatory, for it to show up on the graph. Depending on how long and how much of the inflammatory food was consumed, it took days or sometimes weeks for the effect to wear off. Circles 142 represent that the user 112 had eaten corn chips and tomato two days before bumps D, F, and G appeared on the pain level line 136. The symptom and trigger data tracking system is able to track food reactions even from a one meal use that were not on the strict diet. Corresponding bumps H, I, and J appeared on the pain level line 136.

Circle 144 represents the pain level experience when user 112 purposefully went of the prescribed diet. By the second day the pain level began to really climb. I expected it to go higher and stay higher longer. At that point I was happy to go back on the diet. Circles 146 represent times that user 112 had business lunches where it was not possible to stick to the prescribed diet.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. It will be understood by those skilled in the art that many changes in construction and widely differing embodiments and applications will suggest themselves without departing from the scope of the disclosed subject matter.

1. (canceled)

2. A method for symptom and trigger tracking comprising the steps of:
(a) receiving a plurality of symptom inputs in a symptom logger;
(b) sending by the symptom logger the plurality of symptom inputs to a tracker mobile app running in a user's device;
(c) receiving a plurality of trigger inputs in the tracker mobile app running in the user's device, each of the plurality of trigger inputs representing at least one of a plurality of trigger items;
(d) sending by the tracker mobile app the plurality of symptom inputs received from the system logger, and the plurality of trigger inputs, to a cloud-based database; and
(e) generating by the cloud-based database at least one report based on the plurality of symptom inputs and the plurality of trigger inputs.

3. The method for symptom and trigger tracking according to claim 2 wherein step (a) further comprises the step of:
receiving the plurality of symptom inputs via a plurality of input buttons in the symptom logger, wherein each of the plurality of input buttons represent a different symptom.

4. The method for symptom and trigger tracking according to claim 2 wherein step (a) further comprises the step of:
receiving the plurality of symptom inputs via a plurality of input buttons in the symptom logger, wherein each of the plurality of input buttons represent a different level of effect of a symptom.

5. The method for symptom and trigger tracking according to claim 4 further comprising the step of:
assigning a point value representing a different level of pain to each of the different level of effects; and
averaging all of the point values from each of the plurality of symptom inputs received in the symptom logger during a day to produce a daily score.

6. The method for symptom and trigger tracking according to claim 2 wherein the plurality of trigger items are selected from the group consisting of a food consumed, a diet, a medication taken, an environmental issue such as stress, heat, missing sleep, change of weather, contact with an animal, and a beverage consumed such as an alcoholic beverage, a caffeinated beverage, or other beverage.

7. The method for symptom and trigger tracking according to claim 2 wherein step (e) further comprises the step of:
displaying on a graph a pain level line over several days against a list of trigger food items to determine a correlation between the pain level and the trigger food consumed.

8. The method for symptom and trigger tracking according to claim 2 wherein step (e) further comprises the step of:
displaying on a graph a pain level line over several days against adherence to a diet to determine a correlation between the pain level and the diet.

9. The method for symptom and trigger tracking according to claim 2 further comprising the step of:
calculating by the tracker mobile app a day and time to timestamp each of the plurality of symptom inputs relative to an internal time in the symptom logger associated with each of the plurality of symptom inputs; and
storing the timestamped plurality of symptom inputs in the tracker mobile app and entering each of the timestamped plurality of symptom inputs into a symptom list.

10. The method for symptom and trigger tracking according to claim 2 further comprising the step of:
receiving in the tracker mobile app a new symptom to add, and storing in a current list of symptoms to begin tracking; and
receiving in the tracker mobile app a new trigger item to add, and storing in a current list of trigger items to begin monitoring.

11. The method for symptom and trigger tracking according to claim 2 further comprising the step of:
receiving in the tracker mobile app an answer to at least one journal question, and storing the answer in a list of journal entries.

12. The method for symptom and trigger tracking according to claim 2 further comprising the step of:
receiving in the tracker mobile app a new reminder, and storing the new reminder in a list of reminders.

13. The method for symptom and trigger tracking according to claim 2 wherein step (b) further comprises the step of:
syncing the symptom logger to the tracker mobile app; and
performing step (b) when the symptom logger is within range to communicate wirelessly with the tracker mobile app.

14. The method for symptom and trigger tracking according to claim 2 wherein step (e) further comprises the step of:
sending the at least one report from the cloud-based database to a medical professional via a communications link.

15. An apparatus for logging symptoms comprising:
a top case;
a bottom case;
a cavity formed between the top case and the bottom case;
a plurality of input buttons housed in the cavity, each of the plurality of input buttons extending out of the top case;
a custom designed circuit board housed in the cavity and below the plurality of input buttons, where each of the plurality of input buttons, when pressed, make an electrical connection with the custom designed circuit board, and each of the electrical connections are stored in the custom designed circuit board as a datum representing a predefined symptom.

16. The apparatus for logging symptoms according to claim 15 wherein the custom designed circuit board further comprises:
an onboard processor; and
a connector for receiving a cable, wherein the onboard processor via the cable receives programming for the predefined symptoms; and
a communications module for communicating with a user's device such that when the apparatus for logging symptoms is within range of the user's device, the communications module transfers all of the stored datums representing the predefined symptoms to the user's device.

17. The apparatus for logging symptoms according to claim 16 wherein the user's device is a smartphone, a tablet, or other suitable device capable of running a tracker mobile app that receives the stored datums representing the predefined symptoms.

18. The apparatus for logging symptoms according to claim 15 wherein the predefined symptoms are at least one of a plurality of different symptoms.

19. The apparatus for logging symptoms according to claim 15 wherein the predefined symptoms are a single symptom having of a plurality of levels of effect.

20. A system for symptom and trigger tracking comprising:
a symptom logger;
a plurality of symptom inputs received in the symptom logger;
a user's device;
a mobile tracker app running in the user's device;
a plurality of trigger inputs received in the user's device through the mobile tracker app;
a first wireless connection between the symptom logger and the user's device for sending the plurality of symptom inputs from the symptom logger to the mobile tracker app running in the user's device;
a cloud-based database; and
a second wireless connection between the user's device and the cloud-based database for sending the plurality of symptom inputs and the plurality of trigger inputs from the mobile tracker app running in the user's device to the cloud-based database.

21. The system for symptom and trigger tracking according to claim 20 further comprising:
at least one report based on the plurality of symptom inputs and the plurality of trigger inputs generated by the cloud-based database; and
a communications link emanating from the cloud-based database for sending out the at least one report from the cloud-based database.
